(19) United States
(12) Patent Application Publication
(10) Pub. No.: US 2025/0345254 A1
(43) Pub. Date: Nov. 13, 2025

(54) FORMULATIONS AND USES OF RESVERATROL

(71) Applicant: DANSTAR FERMENT AG, Zug (CH)

(72) Inventors: Marcia DA SILVA PINTO, Reinach (CH); Jérôme DE LA BAUME, Reinach (CH); Clare DODGSHON, Reinach (CH)

(21) Appl. No.: 18/693,766

(22) PCT Filed: Sep. 20, 2022
(86) PCT No.: PCT/EP2022/076107

§ 371 Date: Mar. 20, 2024

Related U.S. Application Data
(60) Provisional application No. 63/246,242, filed on Sep. 20, 2021.
(60) Provisional application No. 63/252,892, filed on Oct. 6, 2021.

(51) Int. Cl. A61K 8/34; A61Q 19/08

(52) U.S. Cl. CPC A61K 8/347; A61Q 19/08; A61K 2800/88

(57) ABSTRACT

The disclosure provides methods of improving skin health parameters and/or reducing signs of skin aging comprising administering to a human subject in need thereof an effective amount of resveratrol.

### FIELD

This disclosure relates to novel uses of resveratrol, e.g., for enhancing skin health, condition and signs of aging.

### BACKGROUND

Resveratrol (3,5,4′-trihydroxy-stilbene) is a polyphenol phytoalexin that plants synthesise in response to external stressors. It exists in two isomeric forms, cis and trans; the trans-form being biologically active. As well as being found in numerous plant species, resveratrol is present in red wine, grapes, dark chocolate, peanuts and berries, and is also used in topical and oral formulations for its antioxidant activity.

Visible signs of skin aging include the accumulation of wrinkles (fine lines and deep lines), dryness, sagging, translucency, uneven pigmentation and visible pores. While intrinsic aging is primarily dependent on genetic processes, extrinsic factors such as lifestyle choices, sun exposure, and pollution accelerate skin aging significantly. Moreover, disruption of the skin barrier may result in adverse health consequences such as stimulating inflammatory dermatosis and allowing the penetration of microbial pathogens into the skin, potentially leading to elevated inflammation in the systemic circulation. There is a need for improved approaches to improving skin function and reducing signs of skin aging.

### SUMMARY

The disclosure provides novel methods and kits for improving skin health parameters and/or signs of skin aging, using resveratrol. In particular, the disclosure provides methods for oral administration and/or topical application of resveratrol to improve skin health parameters and/or signs of skin aging, compared to placebo products, e.g., as measured by:
a. image analysis, e.g., measuring one or more of wrinkles (e.g., fine lines and/or deep lines), uneven pigmentation, skin elasticity, hydration, visible pores, skin tone, sebum, and/or skin temperature), for example, image analysis wherein images are taken under normal, ultraviolet, and polarized light and the image analysis measures pore size, pigmentation, wrinkle number and depth, sebum, moisture, skin elasticity, skin tone, and skin temperature; and/or
b. blood analysis, e.g., measuring one or more of oxidative stress markers and/or blood antioxidant properties, for example total antioxidant capacity, derivatives of reactive oxidative metabolites (DROMs), and/or ratios of oxidized to reduced glutathione (E(h)GSH) and cysteine (E(h)CySH).

### DESCRIPTION

In a first embodiment, the disclosure provides a method (Method 1) of improving skin health parameters and/or reducing signs of skin aging comprising administering to a human subject in need thereof an effective amount of both an oral dosage of resveratrol and a topical formulation of resveratrol. For example, Method 1 includes the following methods:
1.1 Method 1 wherein the resveratrol is 3,5,4′-trihydroxy-trans-stilbene (i.e., trans-resveratrol).
1.2 Method 1 or 1.1 wherein the oral dosage of resveratrol is 50 mg to 1000 mg per day, e.g., 100 mg to 200 mg per day, especially about 150 mg per day.
1.3 Any foregoing method wherein the oral dosage of resveratrol is administered twice daily in divided doses.
1.4 Any foregoing method wherein the oral dosage of resveratrol is 150 mg per day, administered in a divided dose of 75 mg in the morning and 75 mg in the evening.
1.5 Any foregoing method wherein the topical formulation of resveratrol is administered to the skin in an amount of about 2 mg/cm².
1.6 Any foregoing method wherein the topical formulation of resveratrol comprises 0.5 percentage by weight (wt %) to 2.5 wt % resveratrol, especially about 1.5 wt % resveratrol, and is e.g., a cream.
1.7 Any of previous methods 1-1.5 wherein the topical formulation of resveratrol is administered in a daily dosage of resveratrol of 2 mg to 20 mg/100 cm², e.g., 4 mg to 10 mg/100 cm², e.g., about 6 mg/100 cm² of skin.
1.8 Any foregoing method wherein the topical formulation of resveratrol is administered twice daily in divided doses.
1.9 Any foregoing method wherein the topical formulation of resveratrol is administered to the face of the subject.
1.10 Any foregoing method wherein the topical formulation of resveratrol is a cream comprising about 1.5 wt % resveratrol administered to the face of the subject in a dosage of about 1 g in the morning and about 1 g in the evening.
1.11 Any foregoing method wherein
   a. the resveratrol is 3,5,4′-trihydroxy-trans-stilbene;
   b. the oral dosage of resveratrol is 150 mg per day, administered in a divided dose of 75 mg in the morning and 75 mg in the evening; and
   c. the topical formulation of resveratrol is a cream comprising about 1.5 wt % resveratrol administered to the face of the subject in a dosage of about 1 g in the morning and about 1 g in the evening.
1.12 Any foregoing method wherein the duration of resveratrol treatment is at least four weeks, e.g., at least eight weeks.
1.13 Any foregoing method wherein the subject is a female.
1.14 Any foregoing method wherein the subject is over the age of 40.
1.15 Any foregoing method wherein the skin health parameters of the subject improve and/or the signs of skin aging of the subject are reduced compared to the average of a control population which is not receiving resveratrol.
1.16 Any method of 1.15 wherein one or more of the subject's skin health parameters remain the same or worsen less and/or one or more of the subject's signs of skin aging remain the same or increase less between the start and the end of the resveratrol treatment.
1.17 Any of previous methods 1-1.15 wherein the subject has improved skin health parameters and/or reduced signs of skin aging compared to the subject's condition at the start of the resveratrol treatment.
1.18 Any foregoing method wherein the signs of skin aging comprise one or more of increased wrinkles (e.g., increased number and/or depth of fine lines and/or deep lines), increased pore size, increased pigmentation area, decreased sebum, reduced moisture, reduced skin elasticity, reduced skin tone and reduced skin temperature.
1.19 Any foregoing method wherein the signs of skin aging comprise increased number and depth of wrinkles in the eye area.
1.20 Any foregoing method wherein the signs of skin aging are assessed using an image analysis wherein the image analysis measures one, or more, or all, of pore size, pigmentation, wrinkle number and depth, sebum, moisture, skin elasticity, skin tone, and skin temperature, e.g., wherein the image analysis is made using the A-ONE Smart measurement system (Bomtech Electronics Co. Ltd., Seoul, Korea).
1.21 Any foregoing method wherein the signs of skin aging are assessed using an image analysis under normal, ultraviolet, and/or polarized light, e.g., wherein images are taken under normal, ultraviolet, and polarized light.
1.22 Any method of 1.20 or 1.21 wherein the image analysis measures number and depth of wrinkles, pore size, pigmentation area, sebum, moisture, skin elasticity, and skin temperature.
1.23 Any of previous methods 1-1.17 wherein the skin health parameters are assessed using blood analysis, e.g., measuring one or more of oxidative stress markers and/or blood antioxidant properties, for example total antioxidant capacity, derivatives of reactive oxidative metabolites (DROMs), and/or ratios of oxidized to reduced glutathione (E(h)GSH) and cysteine (E(h)CySH).

In another embodiment the disclosure provides resveratrol for use in improving skin health parameters and/or reducing signs of skin aging in a human subject in need thereof, wherein the human subject receives an effective amount of both an oral dosage of resveratrol and a topical formulation of resveratrol, e.g., in accordance with any of above methods 1-1.23.

In another embodiment the disclosure provides the use of resveratrol in the manufacture of a medicament in the form of an oral dosage formulation and a topical formulation for improving skin health parameters and/or reducing signs of skin aging in a human subject in need thereof, e.g., in accordance with any of above methods 1-1.23.

In another embodiment the disclosure provides a kit comprising both an oral dosage of resveratrol and a topical formulation of resveratrol, together with instructions for use, e.g., in accordance with any of above methods 1-1.23.

The term “about” placed before a numerical value “X” refers in the current application to an interval extending from X minus 10% of X to X plus 10% of X.

According to the present invention resveratrol may be administered orally and/or topically as single active ingredient or in combination with other active ingredients, especially as single active ingredient.

In a second embodiment, the disclosure provides a method (Method 2) of improving skin health parameters and/or reducing signs of skin aging comprising administering to a human subject in need thereof an effective amount of resveratrol as an oral dosage of resveratrol. For example, Method 2 includes the following methods:

2.1 Method 2 wherein the resveratrol is 3,5,4′-trihydroxy-trans-stilbene.

2.2 Method 2 or 2.1 wherein the oral dosage of resveratrol is 50 mg to 1000 mg per day, e.g., 100 mg to 200 mg per day, especially about 150 mg per day.

2.3 Any foregoing method wherein the oral dosage of resveratrol is administered twice daily in divided doses.

2.4 Any foregoing method wherein the oral dosage of resveratrol is 150 mg per day, administered in a divided dose of 75 mg in the morning and 75 mg in the evening.

2.5 Any foregoing method wherein the resveratrol is 3,5,4′-trihydroxy-trans-stilbene; and the oral dosage of resveratrol is 150 mg per day, administered in a divided dose of 75 mg in the morning and 75 mg in the evening.

2.6 Any foregoing method wherein the duration of resveratrol treatment is at least four weeks, e.g., at least eight weeks.

2.7 Any foregoing method wherein the subject is a female.

2.8 Any foregoing method wherein the subject is over the age of 40.

2.9 Any foregoing method wherein the skin health parameters of the subject improve and/or the signs of skin aging of the subject are reduced compared to the average of a control population which is not receiving resveratrol.

2.10 Any method of 2.9 wherein one or more of the subject's skin health parameters remain the same or worsen less and/or one or more of the subject's signs of skin aging remain the same or increase less between the start and the end of the resveratrol treatment.

2.11 Any of previous methods 2-2.9 wherein the subject has improved skin health parameters and/or reduced signs of skin aging compared to the subject's condition at the start of the resveratrol treatment.

2.12 Any foregoing method wherein the signs of skin aging comprise one or more of increased wrinkles (e.g., increased number and/or depth of fine lines and/or deep lines), increased pore size, increased pigmentation area, decreased sebum, reduced moisture, reduced skin elasticity, reduced skin tone and reduced skin temperature.

2.13 Any foregoing method wherein the signs of skin aging comprise increased number and depth of wrinkles in the eye area.

2.14 Any foregoing method wherein the signs of skin aging are assessed using an image analysis wherein the image analysis measures one, or more, or all, of pore size, pigmentation, wrinkle number and depth, sebum, moisture, skin elasticity, skin tone, and skin temperature, e.g., wherein the image analysis is made using the A-ONE Smart measurement system (Bomtech Electronics Co. Ltd., Seoul, Korea).

2.15 Any foregoing method wherein the signs of skin aging are assessed using an image analysis under normal, ultraviolet, and/or polarized light, e.g., wherein images are taken under normal, ultraviolet, and polarized light.

2.16 Any method of 2.14 or 2.15 wherein the image analysis measures number and depth of wrinkles, pore size, pigmentation area, sebum, moisture, skin elasticity, and skin temperature. 2.17 Any of previous methods 2-2.11 wherein the skin health parameters are assessed using blood analysis, e.g., measuring one or more of oxidative stress markers and/or blood antioxidant properties, for example total antioxidant capacity, derivatives of reactive oxidative metabolites (DROMs), and/or ratios of oxidized to reduced glutathione (E(h)GSH) and cysteine (E(h)CySH).

In another embodiment the disclosure provides resveratrol for use in improving skin health parameters and/or reducing signs of skin aging in a human subject in need thereof, wherein the human subject receives an effective amount of an oral dosage of resveratrol, e.g., in accordance with any of above methods 2-2.17.

In another embodiment the disclosure provides the use of resveratrol in the manufacture of a medicament in the form or an oral dosage formulation for improving skin health parameters and/or reducing signs of skin aging in a human subject in need thereof, e.g., in accordance with any of above methods 2-2.17.

In a third embodiment, the disclosure provides a method (Method 3) of improving skin health parameters and/or reducing signs of skin aging comprising administering to a human subject in need thereof an effective amount of resveratrol as a topical formulation of resveratrol. For example, Method 3 includes the following methods:
3.1 Method 3 wherein the resveratrol is 3,5,4′-trihydroxy-trans-stilbene.
3.2 Method 3 or 3.1 wherein the topical formulation of resveratrol is administered to the skin in an amount of about 2 mg/cm².
3.3 Any foregoing method wherein the topical formulation of resveratrol comprises 0.5 wt % to 2.5 wt % resveratrol, especially about 1.5 wt % resveratrol, and is e.g., a cream.
3.4 Any of previous methods 3-3.2 wherein the topical formulation of resveratrol is administered in a daily dosage of or resveratrol of 2 mg to 20 mg/100 cm², e.g., 4 mg to 10 mg/100 cm², e.g., about 6 mg/100 cm² of skin.
3.5 Any foregoing method wherein the topical formulation of resveratrol is administered twice daily in divided doses.
3.6 Any foregoing method wherein the topical formulation of resveratrol is administered to the face of the subject.
3.7 Any foregoing method wherein the topical formulation of resveratrol is a cream comprising about 1.5 wt % resveratrol administered to the face of the subject in a dosage of about 1 g in the morning and about 1 g in the evening.
3.8 Any foregoing method wherein
   a. the resveratrol is 3,5,4′-trihydroxy-trans-stilbene; and
   b. the topical formulation of resveratrol is a cream comprising about 1.5 wt % resveratrol administered to the face of the subject in a dosage of about 1 g in the morning and about 1 g in the evening.
3.9 Any foregoing method wherein the duration of resveratrol treatment is at least four weeks, e.g., at least eight weeks.
3.10 Any foregoing method wherein the subject is a female.
3.11 Any foregoing method wherein the subject is over the age of 40.
3.12 Any foregoing method wherein the skin health parameters of the subject improve and/or the signs of skin aging of the subject are reduced compared to the average of a control population which is not receiving resveratrol.
3.13 Any method of 3.12 wherein one or more of the subject's skin health parameters remain the same or worsen less and/or one or more of the subject's signs of skin aging remain the same or increase less between the start and the end of the resveratrol treatment.
3.14 Any of previous methods 3-3.12 wherein the subject has improved skin health parameters and/or reduced signs of skin aging compared to the subject's condition at the start of the resveratrol treatment.
3.15 Any foregoing method wherein the signs of skin aging comprise one or more of increased wrinkles (e.g., increased number and/or depth of fine lines and/or deep lines), increased pore size, increased pigmentation area, decreased sebum, reduced moisture, reduced skin elasticity, reduced skin tone and reduced skin temperature.
3.16 Any foregoing method wherein the signs of skin aging comprise increased number and depth of wrinkles in the eye area.
3.17 Any foregoing method wherein the signs of skin aging are assessed using an image analysis wherein the image analysis measures one, or more, or all, of pore size, pigmentation, wrinkle number and depth, sebum, moisture, skin elasticity, skin tone, and skin temperature, e.g., wherein the image analysis is made using the A-ONE Smart measurement system (Bomtech Electronics Co. Ltd., Seoul, Korea).
3.18 Any foregoing method wherein the signs of skin aging are assessed using an image analysis under normal, ultraviolet, and/or polarized light, e.g., wherein images are taken under normal, ultraviolet, and polarized light.
3.19 Any method of 3.17 or 3.18 wherein the image analysis measures number and depth of wrinkles, pore size, pigmentation area, sebum, moisture, skin elasticity, and skin temperature.
3.20 Any of previous methods 3-3.14 wherein the skin health parameters are assessed using blood analysis, e.g., measuring one or more of oxidative stress markers and/or blood antioxidant properties, for example total antioxidant capacity, derivatives of reactive oxidative metabolites (DROMs), and/or ratios of oxidized to reduced glutathione (E(h)GSH) and cysteine (E(h)CySH).

In another embodiment the disclosure provides resveratrol for use in improving skin health parameters and/or reducing signs of skin aging in a human subject in need thereof, wherein the human subject receives an effective amount of a topical formulation of resveratrol, e.g., in accordance with any of above methods 3-3.20.

In another embodiment the disclosure provides the use of resveratrol in the manufacture of a medicament in the form of a topical formulation for improving skin health parameters and/or reducing signs of skin aging in a human subject in need thereof, e.g., in accordance with any of above methods 3-3.20.

### Example 1—Clinical Trial

A double-blind, randomised, placebo-controlled clinical study is carried out as follows, with an 8-week participation and 4 groups:
Investigational product: trans-Resveratrol (in this Example section referred to as “resveratrol” or “Resveratrol”).
Dose: 75 mg of resveratrol twice daily (morning and evening) in a single capsule with water and/or 1 g of cream (containing 1.5 wt % of Resveratrol) applied topically to the face twice daily (morning and evening)
Duration: 8 weeks
Proposed number of participants: 140

The purpose of the study is to assess the effectiveness of resveratrol for skin health parameters and signs of skin aging compared to placebo in otherwise healthy females aged over 40 years old. The study is designed to show that oral administration and/or topical application of resveratrol improves skin health parameters and signs of skin aging, including wrinkles (fine and/or deep lines), uneven pigmentation, skin elasticity, hydration, visible pores, skin tone, sebum, skin temperature and/or oxidative stress markers and blood antioxidant properties, compared to the placebo products.

Resveratrol can be produced according to processes known in the art. Preferably the resveratrol used in the clinical trial according to the present invention is produced by fermentation using e.g., genetically modified host cells (especially yeast cells), in particular as described in WO 2006/089898 and WO 2016/180956, and wherein the so produced resveratrol can be purified as described for example in WO 2018/141798. In particular, the resveratrol used in the clinical trial according to the present invention is Veri-te™ resveratrol (Food Grade >98%) from Evolva SA, Switzerland.

The participants are divided into the following four study groups:

Group 1-Resveratrol Active treatment oral and topical: The proposed number of participants in this group is 35. Participants are instructed to take 1 capsule (75 mg of Resveratrol) with water 2 times per day (1 capsule in the morning and one capsule in the evening) and apply 1 g amount of cream (containing 1.5 wt % of Resveratrol) to the face twice daily (once in the morning and once in the evening) after cleansing the face and before applying moisturiser.

Composition of the resveratrol capsules:

| Ingredient | Description | [mg] | [%] |
|---|---|---|---|
| Active | Veri-te ™ resveratrol >98% | 75 | 16.55 |
| Additive | CMC (carboxymethyl cellulose) | 275 | 60.67 |
| Additive | MAGNESIUM STEARATE | 5.5 | 1.21 |
| Additive | AEROSIL ® 200 (silicon dioxide) | 2.75 | 0.61 |
| Capsule | While vegetable capsules “0” | 95 | 20.96 |
| | (HPMC) | | |

| | | |
|---|---|---|
| Total | 453 | 100 |

Composition of the resveratrol cream:

| Ingredient | CAS No. | Wt % |
|---|---|---|
| Veri-te ™ resveratrol >98% | | 1.500% |
| Water | 7732-18-5 | 68.690% |
| Caprylic/capric triglyceride | 73398-61-5 | 5.000% |
| Isopropyl myristate | 110-27-0 | 5.000% |
| Betula alba Juice | 84012-15-7 | 4.615% |
| Glycerin | 56-81-5 | 2.480% |
| Glyceryl stearate | 67701-33-1 | 2.000% |
| Cetearyl alcohol | 67762-27-0 | 2.000% |
| Butylene glycol | 107-88-0 | 1.990% |
| Hydroxyethyl acrylate/Sodium | 211286-86-3 | 1.800% |
| acryloyldimethyl taurate copolymer | | |
| Lauryl glucoside | 110615-47-9 | 1.000% |
| Polyglyceryl-2 dipolyhydroxystearate | 144470-58-6 | 1.000% |
| Benzyl alcohol | 100-51-6 | 0.658% |
| Cetearyl glucoside | 246159-33-1 | 0.500% |
| Prunus amygdalus dulcis oil | 8007-69-0 | 0.500% |
| Chondrus crispus extract | 244023-79-8 | 0.350% |
| Sodium benzoate | 532-32-1 | 0.350% |
| Citric acid | 5949-29-1 | 0.310% |
| Acrylates/Steareth-20 methacrylate | 70879-60-6 | 0.300% |
| copolymer | | |
| Tocopheryl acetate | 7695-91-2 | 0.300% |
| Potassium sorbate | 24634-61-5 | 0.200% |
| Sorbitan isostearate | 71902-01-7 | 0.120% |
| Polysorbate 60 | 9005-67-8 | 0.120% |
| Dehydroacetic acid | 520-45-6 | 0.070% |
| 1,2-Hexanediol | 6920-22-5 | 0.025% |
| Sodium lauryl sulfate | 151-21-3 | 0.005% |

Group 2-Resveratrol Active treatment oral and placebo topical: The proposed number of participants in this group is 35. Participants are instructed to take 1 capsule (75 mg of Resveratrol) with water 2 times per day (morning and evening) and apply the placebo topical product as per the active topical treatment group.

Group 3-Placebo oral and Resveratrol active treatment topical: The proposed number of participants in this group is 35. Participants are instructed to take the placebo oral product as per the active oral treatment group and apply 1 g amount of cream (containing 1.5 wt % of Resveratrol) to the face twice daily (morning and evening) after cleansing the face and before applying moisturiser.

Group 4-Placebo oral and topical: The proposed number of participants in this group is 35. Participants are instructed to take the placebo oral and topical products as per the active oral and topical treatment group.

The placebo oral product consists of carboxymethyl cellulose and magnesium stearate and appears identical to the resveratrol oral product. The placebo topical product is a water-based cream (oil in water emulsion) and made identical to the 1.5 wt % resveratrol cream but without the inclusion of resveratrol. Both the active and placebo products are manufactured to GMP standards.

The inclusion criteria for the participants are as follows:
Females aged >40 years old; Otherwise healthy;
Agree not to change standard skincare routine.

The exclusion criteria are as follows:
Clinically significant medical conditions including, but not limited to, cardiovascular, neurological, psychiatric, renal, gastrointestinal, immunological, endocrine (including uncontrolled diabetes or thyroid disease) or haematological abnormalities that are uncontrolled;
Currently taking resveratrol or using resveratrol cream;
Cosmetic surgery and procedures, including Botox and other injectables, microdermabrasion, laser treatments; Medications for acne or other skin conditions including topical retinoids (Rein-A, Retrieve) oral retinoids such as Isotrtinoin (Roaccutane), benzoyl peroxide, AHA's, serum, chemical peels; Active smokers, nicotine use, alcohol or drug (prescription or illegal substances) abuse;
Chronic past and/or current alcohol use (>14 alcoholic drinks week);
Known pregnant or lactating woman; Allergies or hypersensitivity to resveratrol;
Any condition which in the opinion of the investigator makes the participant unsuitable for inclusion.

The trial is a double-blind, randomised, placebo-controlled, clinical study for an 8-week treatment duration utilising active and placebo arms with baseline data collection. Once enrolled in the trial, participants are randomly allocated to one of the four treatment groups on a 1:4 chance basis. Baseline skin assessment and a fasting blood test will be completed at enrolment. Skin assessment includes the characterization of wrinkles, pore size, pigmentation area, sebum, moisture, skin elasticity and skin temperature. Prior to the skin assessment and photograph being taken, participants will remove all make-up and lipstick using provided make-up removal wipes, hair will be cleared away from the face. The fasting blood sample will measure oxidative stress markers, blood antioxidant properties, resveratrol concentration and metabolites.

Once the participant has completed the baseline measurements, they are instructed to take/apply the allocated product according to the prescribed dosing regimen. During the 8-week study period, participants are asked to attend the study site at midpoint (week 4) and the end of the study (week 8) to repeat baseline measurements. At the end of the study (week 8), participants are asked to complete an exit interview and questionnaire. Participants are required to attend the study site in a fasted state during all visits. At the end of the 8 weeks, participation in the study is considered complete. The outcome measures performed at baseline, week 4 and week 8 are listed in Table 1.

TABLE 1
Outcome Measures

| | | Base- | Week | Week |
|---|---|---|---|---|
| Investigations | Measure | line | 4 | 8 |
| Primary Outcome | | | | |
| Wrinkles | Number and depth of wrinkles | ✓ | ✓ | ✓ |
| | measured on total face on a scale of 1-10 | | | |
| | A-ONE Smart/Photography | | | |

Secondary Outcomes

| | | | | |
|---|---|---|---|---|
| Pore size, pigmentation | A-ONE Smart | ✓ | ✓ | ✓ |
| area, sebum, moisture, | Photographs | | | |
| skin elasticity, skin | | | | |
| temperature | | | | |
| Adverse reactions | Symptoms report, biochemistry | ✓ | ✓ | ✓ |
| Biochemical | Oxidative stress markers, blood | ✓ | ✓ | ✓ |
| Measurements | antioxidant properties, resveratrol | | | |
| | concentration (free), metabolites | | | |
| | (conjugated sulphate and glucuronidase) | | | |
| Questionnaire | Participant self-assessment questionnaire | | ✓ | ✓ |
| | (see Table 2) | | | |
| Participant details | Contact information, preliminary | ✓ | | |
| | screening details, medical assessment | | | |
| | and case history | | | |
| Participant experience | Study exit form | | | ✓ |

| | | |
|---|---|---|
| Safety | Undesirable and unwanted events | At each point of |
| | | contact |

TABLE 2
Questionnaire
Self-assessment Questionnaire
Please rate your agreement with the following statements:
1. I am satisfied with the overall results this product provides

| | | | | |
|---|---|---|---|---|
| Strongly | Disagree | Indifferent | Agree | Strongly |
| disagree | somewhat | | somewhat | agree |

2. My lines and wrinkles are less visible

| | | | | |
|---|---|---|---|---|
| Strongly | Disagree | Indifferent | Agree | Strongly |
| disagree | somewhat | | somewhat | agree |

3. My face appears more firm

| | | | | |
|---|---|---|---|---|
| Strongly | Disagree | Indifferent | Agree | Strongly |
| disagree | somewhat | | somewhat | agree |

4. My skin tone has become more even

| | | | | |
|---|---|---|---|---|
| Strongly | Disagree | Indifferent | Agree | Strongly |
| disagree | somewhat | | somewhat | agree |

5. My skin brightness has increased

| | | | | |
|---|---|---|---|---|
| Strongly | Disagree | Indifferent | Agree | Strongly |
| disagree | somewhat | | somewhat | agree |

6. My skin texture has improved

| | | | | |
|---|---|---|---|---|
| Strongly | Disagree | Indifferent | Agree | Strongly |
| disagree | somewhat | | somewhat | agree |

7. My skin looks refreshed

| | | | | |
|---|---|---|---|---|
| Strongly | Disagree | Indifferent | Agree | Strongly |
| disagree | somewhat | | somewhat | agree |

8. My skin feels more resilient

| | | | | |
|---|---|---|---|---|
| Strongly | Disagree | Indifferent | Agree | Strongly |
| disagree | somewhat | | somewhat | agree |

9. My skin clarity has improved

| | | | | |
|---|---|---|---|---|
| Strongly | Disagree | Indifferent | Agree | Strongly |
| disagree | somewhat | | somewhat | agree |

10. My face feels rejuvenated

| | | | | |
|---|---|---|---|---|
| Strongly | Disagree | Indifferent | Agree | Strongly |
| disagree | somewhat | | somewhat | agree |

11. My skin has a youthful glow/radiance

| | | | | |
|---|---|---|---|---|
| Strongly | Disagree | Indifferent | Agree | Strongly |
| disagree | somewhat | | somewhat | agree |

12. My skin feels more firm

| | | | | |
|---|---|---|---|---|
| Strongly | Disagree | Indifferent | Agree | Strongly |
| disagree | somewhat | | somewhat | agree |

13. My skin looks and feels more hydrated

| | | | | |
|---|---|---|---|---|
| Strongly | Disagree | Indifferent | Agree | Strongly |
| disagree | somewhat | | somewhat | agree |

14. My skin looks more healthy

| | | | | |
|---|---|---|---|---|
| Strongly | Disagree | Indifferent | Agree | Strongly |
| disagree | somewhat | | somewhat | agree |

15. My skin feels more balanced

| | | | | |
|---|---|---|---|---|
| Strongly | Disagree | Indifferent | Agree | Strongly |
| disagree | somewhat | | somewhat | agree |

16. My pores appear less visible

| | | | | |
|---|---|---|---|---|
| Strongly | Disagree | Indifferent | Agree | Strongly |
| disagree | somewhat | | somewhat | agree |

17. My skin feels soothed

| | | | | |
|---|---|---|---|---|
| Strongly | Disagree | Indifferent | Agree | Strongly |
| disagree | somewhat | | somewhat | agree |

We claim:

1. A method of improving skin health parameters and/or reducing signs of skin aging comprising administering to a human subject in need thereof an effective amount of both an oral dosage of resveratrol and a topical formulation of resveratrol.

2. The method of claim 1, wherein the resveratrol is 3,5,4′-trihydroxy-trans-stilbene.

3. The method of claim 1, wherein the oral dosage of resveratrol is 50 mg to 1000 mg per day, e.g., 100 mg to 200 mg per day, especially about 150 mg per day, e.g., wherein the oral dosage of resveratrol is administered twice daily in divided doses, e.g., wherein the oral dosage of resveratrol is 150 mg per day, administered in a divided dose of 75 mg in the morning and 75 mg in the evening.

4. The method of claim 1, wherein the topical formulation of resveratrol is administered to the skin in an amount of about 2 mg/cm², e.g., wherein the topical formulation of resveratrol comprises 0.5 wt % to 2.5 wt % resveratrol, especially about 1.5 wt % resveratrol, and is e.g., a cream, or e.g., wherein the topical formulation of resveratrol is administered in a daily dosage of resveratrol of 2 mg to 20 mg/100 cm², e.g., 4 mg to 10 mg/100 cm², e.g., about 6 mg/100 cm² of skin, e.g., wherein the topical formulation of resveratrol is administered twice daily in divided doses, e.g., wherein the topical formulation of resveratrol is administered to the face of the subject, e.g., wherein the topical formulation of resveratrol is a cream comprising about 1.5 wt % resveratrol administered to the face of the subject in a dosage of about 1 g in the morning and about 1 g in the evening.

5. The method of claim 1, wherein
a. the resveratrol is 3,5,4′-trihydroxy-trans-stilbene;
b. the oral dosage of resveratrol is 150 mg per day, administered in a divided dose of 75 mg in the morning and 75 mg in the evening; and
c. the topical formulation of resveratrol is a cream comprising about 1.5 wt % resveratrol administered to the face of the subject in a dosage of about 1 g in the morning and about 1 g in the evening.

6. The method of claim 1, wherein the duration of resveratrol treatment is at least four weeks, e.g., at least eight weeks.

7. The method of claim 1, wherein the subject is over the age of 40.

8. The method of claim 1, wherein the skin health parameters of the subject improve and/or the signs of skin aging of the subject are reduced compared to the average of a control population which is not receiving resveratrol, or wherein the subject has improved skin health parameters and/or reduced signs of skin aging compared to the subject's condition at the start of the resveratrol treatment.

9. The method of claim 1, wherein the signs of skin aging comprise one or more of increased wrinkles, increased pore size, increased pigmentation area, decreased sebum, reduced moisture, reduced skin elasticity, reduced skin tone and reduced skin temperature, e.g., wherein the signs of skin aging comprise increased number and depth of wrinkles in the eye area.

10. The method of claim 1, wherein the signs of skin aging are assessed using an image analysis wherein the image analysis measures one, or more, or all, of pore size, pigmentation, wrinkle number and depth, sebum, moisture, skin elasticity, skin tone, and skin temperature, e.g., wherein the image analysis is made using the A-ONE Smart measurement system (Bomtech Electronics Co. Ltd., Seoul, Korea).

11. The method of claim 1, wherein the skin health parameters are assessed using blood analysis, e.g., measuring one or more of oxidative stress markers and/or blood antioxidant properties, for example total antioxidant capacity, derivatives of reactive oxidative metabolites (DROMs), and/or ratios of oxidized to reduced glutathione (E(h)GSH) and cysteine (E(h)CySH).

12. A method of improving skin health parameters and/or reducing signs of skin aging comprising administering to a human subject in need thereof an effective amount of resveratrol as an oral dosage of resveratrol or as a topical formulation of resveratrol, wherein the skin health parameters of the subject improve and/or the signs of skin aging of the subject are reduced compared to the average of a control population which is not receiving resveratrol, or the subject has improved skin health parameters and/or reduced signs of skin aging compared to the subject's condition at the start of the resveratrol treatment, e.g., wherein the signs of skin aging are assessed using an image analysis wherein the image analysis measures one, or more, or all, of pore size, pigmentation, wrinkle number and depth, sebum, moisture, skin elasticity, skin tone, and skin temperature, e.g., wherein the image analysis is made using the A-ONE Smart measurement system (Bomtech Electronics Co. Ltd., Seoul, Korea), and e.g, wherein the skin health parameters are assessed using blood analysis, e.g., measuring one or more of oxidative stress markers and/or blood antioxidant properties, for example total antioxidant capacity, derivatives of reactive oxidative metabolites (DROMs), and/or ratios of oxidized to reduced glutathione (E(h)GSH) and cysteine (E(h)CySH).

13. (canceled)

14. A kit comprising both an oral dosage of resveratrol and a topical formulation of resveratrol, together with instructions for use according to the method of claim 1.
